(19) United States
(12) Patent Application Publication
(10) Pub. No.: US 2019/0072567 A1
(43) Pub. Date: Mar. 7, 2019

(54) METHOD OF DETECTING LC3B EXPRESSION IN EXOSOMES EXTRACTED FROM LIQUID BIOLOGICAL SAMPLES

(71) Applicant: University of Cincinnati, Cincinnati, OH (US)

(72) Inventors: Ola Gaber, Cincinnati, OH (US); Nagla Abdel-Karim, Montgomery, OH (US); Ihab Eldessouki, Cincinnati, OH (US)

(21) Appl. No.: 16/118,671

(22) Filed: Aug. 31, 2018

Related U.S. Application Data
(60) Provisional application No. 62/553,170, filed on Sep. 1, 2017.

(51) Int. Cl. G01N 33/68

(52) U.S. Cl. CPC G01N 33/6893; G01N 2333/4703

(57) ABSTRACT

Provided herein is a method of detecting microtubule-associated protein 1 light chain 3 (LC3B) expression in a subject as a marker for autophagy, the method comprising: obtaining a liquid biological sample from the subject; isolating exosomes from the liquid biological sample; and detecting LC3B expression in the isolated exosomes. Also provided is a method of detecting LC3B expression as a marker for autophagy in a subject suffering from a solid malignancy, comprising: obtaining a peripheral blood sample from the subject; isolating exosomes from the peripheral blood sample; and detecting LC3B expression in the isolated exosomes, wherein the method does not include obtaining a biopsy of the solid malignancy.

### CROSS REFERENCE TO RELATED APPLICATIONS

This application claims priority to U.S. Provisional Application Ser. No. 62/553,170, filed Sep. 1, 2017, which application is hereby incorporated by reference in its entirety.

### TECHNICAL FIELD

This disclosure relates to the field of detecting protein expression in exosomes. More specifically, this disclosure relates to detecting expression of microtubule-associated protein 1 light chain 3 B (LC3B) in exosomes extracted from a liquid biological sample obtained from a subject, as a marker for autophagy.

### BACKGROUND

Autophagy is a catabolic process utilized by cells to counter starvation, recycle nutrients, and remove unwanted or damaged cellular structures. Autophagy can occur constitutively or in response to cellular stressors. Three autophagy pathways have been defined: (1) macroautophagy, wherein double membrane vesicles (e.g., autophagosomes) sequester whole cytosolic regions and lysosomes fuse with the autophagosomes to degrade contents; (2) chaperone-mediated autophagy (CMA), a more selective process wherein cytosolic proteins to be degraded are specifically recognized by chaperone hsc70, and LAMP-2A on the surface of lysosomes acts as a receptor for hsc70 to assist in engulfing proteins for degradation; and (3) microautophagy, wherein lysosomes engulf small cytosolic components directly.

Autophagosomes are double membrane vesicles that mediate the process of autophagy. Two proteins are integral in formation of autophagosomes: microtubule-associated protein 1 light chain 3 (LC3), a mammalian homolog of yeast autophagy-related gene 8; and SQSTM1/p62, a cargo protein. LC3s are structural proteins of autophagosomal membranes, of which three family members have been identified (A, B, C). LC3B has been recognized in the art as a marker of autophagy in cells. LC3B-I is present in the cytosol. When covalently linked with phosphatidylethanolamine, LC3B-I is converted to LC3B-II and is present in the autophagosome membrane during the autophagy cycle.

The role of autophagy in cancer has not been clearly defined, although correlations have been observed. Autophagy is believed to play a role in assisting cancer cells in evading cancer treatments. Accordingly, inhibition of autophagy is believed to be a potential direction for cancer therapy.

Exosomes are small vesicles ranging from 30-100 nm in diameter, found in nearly all eukaryotic fluids. Cells secrete exosomes into their extracellular environment. They are formed as intraluminal vesicles (ILVs) by inward budding of the limiting membrane into the lumen of late endosomes or multivesicular bodies (MVBs). Upon fusion of MVBs with the plasma membrane, the vesicles are released as exosomes and enter body fluids, such as blood, urine, and ascites. Ludwig, et al., Exosomes: Small vesicles participating in intercellular communication, Int'l. J. of Biochem. & Cell Bio. 44(1): 11-15 (2012). Exosomes participate in a variety of cell functions, including transfer of DNA, RNA, and proteins between cells and intercellular communication. Exosomes are released in high quantities from rapidly growing cells, including cancer cells.

Studies suggest a relationship between autophagy and exosome secretion. The endosomal sorting complexes required for transport (ESCRT) machinery plays a role in the formation of ILVs. ESCRT mutants were found to lack the ability to fuse autophagosomes with endolysosomes, leading to accumulation of autophagosomes and increased exosome secretion. Further, autophagy inducers have been shown to reduce exosome release.

Cancer researchers have recognized the benefit of monitoring autophagy in a subject in order to better understand the cellular processes underpinning cancer development and response to treatment. However, current methods rely on invasive biopsy of cancer tissues, particularly solid malignancies, and detection of autophagy markers by immunohistochemistry, electron microscopy, fluorescence imagining, immunoblotting, and the like.

A need exists for less-invasive methods of monitoring autophagy in cancer patients.

### SUMMARY

The present disclosure is directed to a liquid biopsy of a biological sample obtained from a subject to detect autophagy related proteins, including LC3B-I and LC3B-II, as markers for autophagy in the subject.

In one embodiment, a method is provided for detecting microtubule-associated protein 1 light chain 3 B (LC3B) expression associated with autophagy in a subject, the method comprising: obtaining a liquid biological sample from the subject; isolating exosomes from the liquid biological sample; and detecting LC3B expression in the isolated exosomes.

In another embodiment, a method is provided for detecting microtubule-associated protein 1 light chain 3 B (LC3B) expression associated with autophagy in a subject suffering from a solid malignancy, the method comprising: obtaining a peripheral blood sample from the subject; isolating exosomes from the peripheral blood sample; and detecting LC3B expression in the isolated exosomes, wherein the method does not include obtaining a biopsy of the solid malignancy.

These and other objects, features, embodiments, and advantages will become apparent to those of ordinary skill in the art from a reading of the following detailed description and the appended claims.

### BRIEF DESCRIPTION OF THE DRAWINGS

FIG. 1. (A) Positive and negative controls from cells treated with chloroquine are shown. Cells show an increase in LC3B-II band thickness with chloroquine treatment. The exosomes blot shows a clear LC3B-II band with chloroquine treatment, but lacks LC3B-I bands. Clarity of the LC3B-II band demonstrates that LC3B expression in exosomes is suitable for use as a marker of autophagy in cells. (B) Treated U251 cells and HEK293 cells and exosomes were run on the same gel, with exosomes extracted from patient #8, cycle 1. Cell lines showed different band thickness in LC3B isoforms with same chloroquine concentration and time point, suggesting different baseline autophagy in different cell lines. Patient plasma showed same pattern of migration as tested cell lines, which was affected by timing relative to treatment administration.

FIG. 2. Exosomes extracted from chloroquine (CQ)+gemcitabine (Gem) treated MiaPaCa2 cells did not show any additional increase in band thickness of LC3B-II. Treatment of MiaPaCa2 cells with Gem alone yielded significantly less intense bands for both forms of LC3B. Decrease in band intensity with combination treatment may be attributed to inducement of autophagy by Gem.

FIG. 3. LC3B expression in exosomes extracted from MiaPaCa2 cells increased in intensity when concentration of CQ was increased, when administered in combination with Gem. Results indicate that Gem aids in decreasing LC3B intensity in both forms.

FIG. 4. Western blot analysis for patient who underwent 4 cycles of Gem/Carb treatment regimen+CQ. At end of 4 cycles, no clear bands for LC3B-II expression were visible. Results indicate that the inhibitory effect of CQ was not sufficient to antagonize the stimulatory effect of the chemotherapy protocol administered to the patient. LC3B-II expression was below detectable threshold.

FIG. 5. Western blot analysis for patient receiving 100 mg CQ per day. Patient demonstrated LC3B-II expression in third cycle of treatment, first detected at 6h, with peak detection on day 15.

FIG. 6. Western blot analysis demonstrating presence of CD9, a control, in extracted exosomes. Results show variation in amounts of exosomes in patient samples. LC3B was not detected in samples that showed no bands for CD9.

FIG. 7. LC3B expression in patient 12 is shown via western blot analysis. LC3B-II expression was evident at 24 h during first cycle of treatment.

FIG. 8. Annexin V assay to determine the effect of Gem and CQ on HEK293 cells. (A) Non-treated cells showed 46.9% live cells (Q3) compared to 10% apoptotic cells and 36.5% of the cells in early apoptosis with 6.6% necrosis. (B) HEK293 cells treated with 20 μM Gem: Percentage of live cells decreased to 29.3% and early apoptotic cells increased to 56.3%. (C) HEK293 cells treated with 10 μM CQ: percentage of apoptotic cells increased to 17% with no significant difference in other groups compared to (B). (D) HEK293 cells treated with 20 μM CQ: doubling CQ concentration decreased both live and necrotic cells in favor of early apoptotic and apoptotic cells. (E)-(F) Combinations of 20 μM Gem in combination with either 10 μM CQ (E) or 20 μM CQ (F) showed both treatments increased apoptotic cells compared to 20 μM Gem alone. Although early apoptotic and apoptotic cells were similar to CQ-only treated cells, the rise in necrotic cells was notably high in the combined therapies, for both concentrations of CQ.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided herein.

While the following terms are believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

It is to be further understood that where descriptions of various embodiments use the term “comprising,” and/or “including” those skilled in the art would understand that in some specific instances, an embodiment can be alternatively described using language “consisting essentially of” or “consisting of.”

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, “subject” refers to a mammalian subject, particularly a human subject. In some embodiments, the subject is suffering from cancer. In certain embodiments, the subject is suffering from a solid malignancy. In specific embodiments, the solid malignancy is an advanced stage malignancy or a recurrent malignancy. In embodiments, the terms “subject” and “patient” are used interchangeably.

The term “liquid biological sample,” as used herein, refers to a biological sample obtained from a patient, from which exosomes can be extracted. In one embodiment, the liquid biological sample comprises peripheral blood, ascites, and urine. In another embodiment, the liquid biological sample comprises plasma obtained from a patient's blood sample. In certain embodiments, the liquid biological sample is obtained in a manner less-invasive than a biopsy of a solid malignancy, e.g., by a simple blood draw or urine collection.

The combination of gemcitabine (Gem) and carboplatin (Carb) is a standard chemotherapy regimen indicated for treatment of advanced solid tumors. Studies have shown a correlation between gemcitabine and induction of autophagy, perhaps due to increased cellular stress. Chloroquine (CQ) is a 4-alkylamino substituted quinoline family member that inhibits autophagy by blocking the fusion of autophagosomes and lysosomes, thereby inhibiting the final stages of autophagy. The accumulation of autophagosomes leads to an increase in release of the unwanted cell materials via exosomes. Disclosed herein are methods employing western blotting to detect LC3B-I and II isoforms in exosomes extracted from subject plasma. The methods described herein have application as a liquid biopsy to monitor autophagy in a subject, particularly a human patient.

A Gem/Carb chemotherapy regimen was administered to 12 solid malignancy patients for whom Gem/Carb is a reasonable standard-of-care treatment. CQ in varying concentrations was administered in combination with Gem/Carb as lysosomal inhibitor. The resulting increase in exosome secretion by cells was examined by western blotting for the detection of LC3B in liquid biological samples obtained from the patients. The 12 study participants received Gem/Carb+CQ, wherein CQ administration began 7 days prior (i.e., day −7) to Gem/Carb treatment. Solid malignancies included hepatocellular carcinoma, cholangiocarcinoma, colorectal carcinoma, gastro-intestinal stromal tumor, non-small cell lung cancer, small cell lung cancer, and urothelial cancer. All were patients were diagnosed with advanced stage or recurrent malignancy. Results of western blotting validated the premise that LC3B-I is detectable in exosomes extracted from patient blood, thus providing a less-invasive method for assessing autophagy in a patient, particularly a cancer patient, without the need for an invasive biopsy of solid tumor tissue.

Accordingly, in one embodiment, a method of detecting microtubule-associated protein 1 light chain 3 B (LC3B) expression as a marker for autophagy in a subject is provided, the method comprising: obtaining a liquid biological sample from the subject; isolating exosomes from the liquid biological sample; and detecting LC3B expression in the isolated exosomes. In embodiments, the liquid biological sample is selected from the group consisting of peripheral blood, ascites, and urine. In a specific embodiment, the liquid biological sample is peripheral blood. In another embodiment, the liquid biological sample is plasma, e.g., plasma obtained from a peripheral blood sample. A particular advantage of the disclosed methods is the ability to monitor autophagy in a patient diagnosed with a solid malignancy by assaying LC3B expression in exosomes isolated from a liquid biological sample from the patient, without the need for a biopsy of the solid malignancy.

Various methods of extracting exosomes from biological samples are known in the art. In certain embodiments, isolating exosomes from the liquid biological sample comprises: centrifuging the liquid biological sample to provide a supernatant and a pellet comprising cells and/or cellular debris; treating the supernatant with an exosome extraction reagent to precipitate the exosomes; centrifuging the treated supernatant to provide a pellet comprising the exosomes; and resuspending the exosomes in phosphate buffered saline. In other embodiments, exosomes are extracted by ultracentrifugation, which methods are understood in the art.

In certain embodiments, LC3B expression is detected via western blotting. Methods of carrying out western blot assays are well known in the art. See, for example, Gomez-Sanchez, et al., Routine western blot to check autophagic flux: cautions and recommendations, Anal. Biochem. 477: 13-20 (2015). In certain embodiments, LC3B expression is detected with anti-LC3B monoclonal or polyclonal antibodies. In certain embodiments, the antibodies are goat, rabbit, or mouse antibodies. In a specific embodiment, the antibody is a monoclonal rabbit anti-LC3B antibody.

In certain embodiments, the methods comprise a further step of quantifying LC3B expression in the isolated exosomes. Methods of quantifying protein expression are known in the art. See, for example, Zhang, et al., Methods for the detection of autophagy in mammalian cells, Curr. Protoc. Toxicol. 69: 20.12.1-20.12.16 (2016).

LC3B is detected in exosomes extracted from patient liquid biological samples as a marker for autophagy in patient cells. In certain embodiments, detecting LC3B expression comprises detecting expression of one or more of LC3B-I and LC3B-II.

In another embodiment, a method of detecting microtubule-associated protein 1 light chain 3 B (LC3B) expression as a marker for autophagy in a subject suffering from a solid malignancy is provided, the method comprising: obtaining a peripheral blood sample from the subject; isolating exosomes from the peripheral blood sample; and detecting LC3B expression in the isolated exosomes, wherein the method does not include obtaining a biopsy of the solid malignancy.

In embodiments, isolating exosomes from the peripheral blood sample comprises: centrifuging the peripheral blood sample to provide a supernatant and a pellet comprising cells and cellular debris; treating the supernatant with an exosome extraction reagent to precipitate the exosomes; centrifuging the treated supernatant to provide a pellet comprising the exosomes; and resuspending the exosomes in phosphate buffered saline.

In certain embodiments, LC3B expression is detected via western blotting. In certain embodiments, LC3B expression is detected with anti-LC3B monoclonal or polyclonal antibodies. In certain embodiments, the antibodies are goat, rabbit, or mouse antibodies. In a specific embodiment, the antibody is a monoclonal rabbit anti-LC3B antibody. In certain embodiments, the methods comprise a further step of quantifying LC3B expression in the isolated exosomes. As described in other embodiments, detecting LC3B expression comprises detecting expression of one or more of LC3B-I and LC3B-II.

### EXAMPLES

The following detailed methodology and materials are set forth to support and illustrate particular aspects and embodiments of the invention, and should not be construed as limiting the scope thereof.

### Example 1. Materials and Methods

### Study Population

The study included 12 patients who were included within cohorts 1-3 of the approved phase I clinical trial (ClinicalTrials.gov Identifier NCT02071537). Patients were diagnosed with advanced solid malignancies of various histopathology who did not have other treatment options or who qualified to receive carboplatin/gemcitabine as part of their therapeutic options. All subjects were consented before treatment and sample collection. All patients were heavily pretreated with anti-cancer therapeutics, with the exception of one patient who was enrolled to receive therapy with carboplatin/gemcitabine as part of the standard-of-care, in addition to the study CQ.

### Study Population Treatment

All 12 patients received Gem/Carb for from one to four cycles at standard doses, wherein the first three patients of cohort one received Gem 1200 mg/m2 and the remaining three patients of cohort one received 1200 mg/m² (Gem dose decreased in some patients due to heavy pretreatment, in order to avoid high toxicity profile). Carboplatin was received as area under the curve (AUC) 5 and dose modifications were permitted. The clinical trial is ongoing. The first 6 patients received 50 mg CQ daily. When well tolerated, the dose was increased by 50 mg increments to 100 mg/day (3 patients) and 150 mg/day (3 patients). All candidates began taking CQ orally 7 days prior to chemotherapy (day −7) and continued taking CQ daily throughout four cycles of Gem/Carb. All enrolled subjects had at least one lesion to assess their response according to RECIST Criteria Version 4.1.

### Cell Culturing

Three cell lines were used in the studies disclosed herein. U251, a human glioblastoma cell line, MiaPaCa2, a human pancreatic carcinoma cell line, and HEK293, a human embryonic kidney cell line. A variety of cell lines were selected in order to compare results for cells having different baseline autophagic activities. Cells were maintained in DMEM culture media supplemented with 10% fetal bovine serum (FBS), antimycotic, and L-glutamine. At 40% confluence, culture dishes were washed twice with PBS and media was replaced with 10% exosome depleted FBS (dFBS). DMEM media (50 ml EXO-FBS™ Exosome Depleted FBS media, System Biosciences) and incubated to settle for 24 h. After 24 h, culture dishes were washed with PBS and new dFBS DMEM media was added and treated with chloroquine diphosphate salt (Sigma Aldrich, UK) and incubated. Incubation period was 48 h for western blot on HEK293 plates and 72 h for western blot of U251 cells. Incubation varied according to plate confluency, CQ concentration, and expected number of exosomes secreted by the cell line. Media was collected and used for exosome extraction directly after incubation period concluded.
Exosome Extraction from Patient Plasma

Study participant blood samples were collected at defined time points and centrifuged at 1500×g for 15 minutes. The upper phase (plasma) was collected in new tubes and stored at −80° C. until use. Exosome extraction was carried out using exosome extraction reagent (Total Exosome Precipitation Reagent from plasma, Invitrogen by Thermo-Fisher Scientific) according to manufacturer instructions. Briefly, the exosome isolation reagent enables concentration of intact exosomes from plasma samples by forcing less-soluble components such as vesicles out of solution, allowing collection via low-speed centrifugation. Optionally, Proteinase K is used to eliminate all plasma protein prior to exosome extraction, in order to maximize exosome preparation purity. After a 10 minute treatment with Proteinase K, the exosome isolation reagent is added to the plasma and the solution is incubated for 30 min. at 2-8° C. Precipitated exosomes are recovered by standard centrifugation at 10,000×g for 5 min. at room temperature. Extracted exosomes were suspended in phosphate buffered saline (PBS) and stored at −80° C.

Exosome Extraction from Cell Culture Media

Exosomes were extracted from freshly collected cell culture media using exosome precipitation reagent (Total Exosome Isolation from cell culture media, Invitrogen by Life Technologies) following manufacturer instructions. Briefly, exosome extraction reagent is added to cell culture media and the solution is incubated overnight at 2-8° C. Precipitated exosomes are recovered by standard centrifugation at 10,000×g for 60 min. Extracted exosomes were suspended in phosphate buffered saline (PBS) and stored at −80° C.

### Western Blotting

Detection of LC3B expression in isolated exosomes was completed using western blotting standard protocols. Briefly, gel electrophoresis was used to separate proteins by size and/or structure. Proteins were then transferred to a membrane (polyvinylidene fluoride or nitrocellulose are suitable for use) and blocked with blocking buffer to prevent non-specific antibody binding. Membranes were incubated with anti-LC3B antibody to target LC3B-I and LC3B-II. LI-COR detection (LI-COR Biotechnology, USA) was employed to visualize target proteins. LC3B protein detection was achieved by anti-LC3B rabbit monoclonal antibody (Cell Signaling Inc., USA). CD9 was used as a loading control and was detected using rabbit monoclonal antibody (Cell Signaling Inc., USA). All western blots were run on 5-15% gradient gels after estimating and unifying sample protein content by bicinchoninic acid assay (BCA).

### Flow Cytometry

HEK293 cells were plated in 10 cm culture dishes in 10% FBS DMEM media. Media was replaced with dFBS DMEM once 40% confluence was reached and cells were incubated for 24 h. Cells were treated with the following concentrations of agents: Gem 20 μM concentration (Gem²⁰), CQ 10 μM (CQ¹⁰) and 20 μM (CQ²⁰) concentrations, and in combinations (wherein Gem was administered at 20 μM but CQ concentration varied) as follows: Gem+CQ¹⁰ and Gem+CQ²⁰. Cells were treated in fresh dFBS DMEM media for 16 h. Cells and media were collected, centrifuged, and washed and stained with propedium iodide (PI) and annexin V prior to running samples. Cell cycle analysis was performed using fresh cells on a FACS Calibur (Becton Dickinson) after incubation with 25 μg/ml PI. Cell cycle phases were analyzed with the CellQuest-Pro software program (Becton Dickinson).

### Example 2. Quantification of Autophagy Markers LC3B-I and LC3B-II by Western Blot

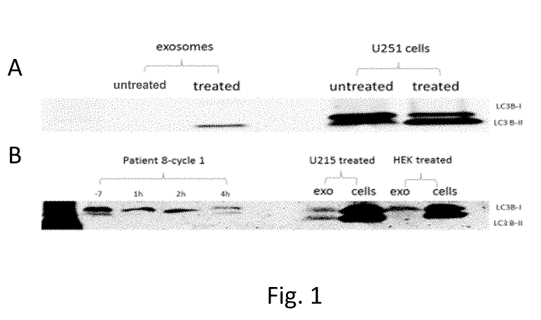

Quantification of LC3B-I and LC3B-II by western blot was compared in U251 cells, exosomes extracted from cell media, patient peripheral blood, and HEK293 cells (FIG. 1). U251 and HEK293 cells were treated with CQ to induce exosome secretion. Both cells and exosomes extracted from cellular media were analyzed. A peripheral blood sample was obtained from a patient undergoing Gem/Carb treatment, with a −7 daily pretreatment with CQ. Exosomes were isolated from the patient's plasma sample.

As shown in FIG. 1(A), left panel, LC3B-II was detected in exosomes isolated from U251 cells treated with CQ. When total cell extracts were tested, LC3B-II was prominent in both CQ-treated and CQ-untreated samples, indicating that isolated exosomes are suitable for use as a surrogate marker to quantify cellular autophagy (FIG. 1(A), right panel). Exosomes extracted from patient plasma also indicated presence of LC3B-I and LC3B-II (FIG. 1(B), left panel), indicating that the technique is sensitive enough to quantify autophagy in plasma. Exosome and whole cell fractions for treated U251 and HEL293 cells are compared in FIG. 1(B), right panel. Differences in band thickness indicate variation in baseline autophagy in different cell lines.

### Example 3. Detecting LC3B in Exosomes Extracted from Cell Lines Treated with Gem, Carb, and Combinations Thereof

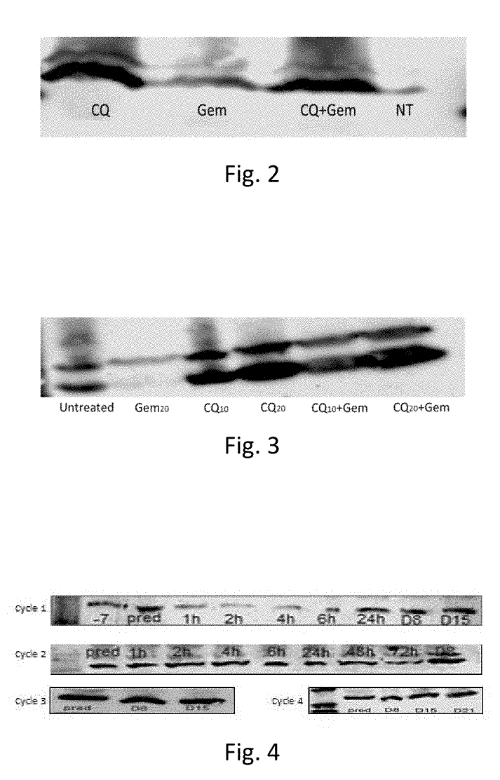

MiaPaCa2 cells were treated with CQ 50 μM, Gemcitabine 20 μM, or a combination of both agents. As shown in the western blot of FIG. 2, all samples showed clear expression for LC3B-II, supporting the premise that different cell lines have different baseline autophagic activity. Exosomes from cells exposed to CQ showed clear expression of LC3B-II. Exosomes from cells exposed to Gem only showed a comparatively less intense band for LC3B-II, which may be attributed to previous reports that Gem is an inducer for autophagy. It is noted that the combination of CQ+Gem yielded an LC3B-II band slightly less intense than that for the CQ-only treated cells. Without being bound by theory, it is believed that the decrease in intensity is due to the autophagy inducing properties of Gem.

Different concentrations of Gem and CQ were analyzed, with results shown in FIG. 3. Intensity of LC3B-II bands for exosomes from cells treated with combinations of Gem and CQ were comparatively less intense compared to CQ-only treated cells, further supporting the premise that Gem induces autophagy, leading to lower expression of LC3B.

### Example 4. Detection of LC3B from Exosomes in Patient Plasma

Blood samples from patients were drawn prior to beginning CQ treatment on day −7. CQ was administered for 7 days beginning on day −7, and Gem/Carb treatment began on day 1. Patients received Gem on days 1 and 8 of each 21 day cycle; patients received Carb on day 1 of each 21 day cycle. Patients participated in up to 4 cycles of treatment. Blood samples were drawn at different time points: pre-dosing with Gem/Carb, 1h, 2h, 4h, 6h, 24h, day 8, and day 15 of the cycle. Some time points were omitted due to unavailability of the patient.

For the first group of patients (n=6), only one patient was able to tolerated the full course of Gem/Carb+CQ. Western blot results for exosomes extracted from that patient's blood are shown in FIG. 4. Three other patients in this group received 2 cycles of therapy prior to stopping treatment. LC3B-II was not detected in the plasma extracted exosomes for the patient who underwent the full course of treatment (FIG. 4). It is believed that the failure to detect LC3B-II is due to the low dose of CQ administered to the patient (50 mg/day).

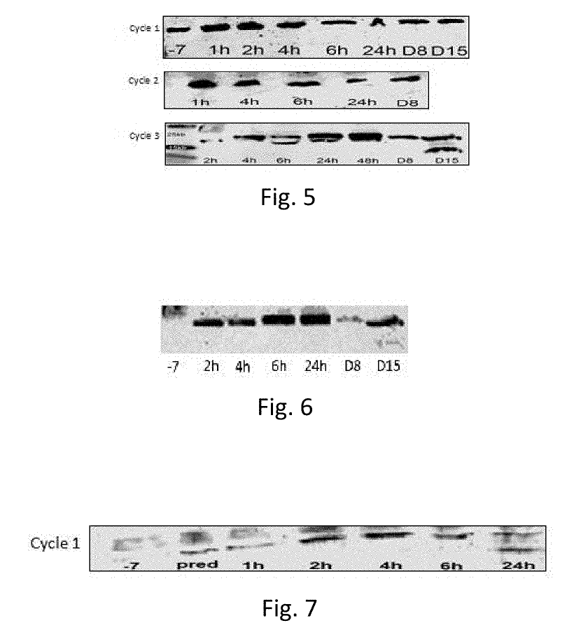

A second group of patients (n=3) received a dose of 100 mg/day CQ, in addition to the Gem/Carb regimen. Two patients completed all 4 cycles of treatment. Of these patients, one patient expressed both forms of LC3B-I and LC3B-II in western blotting (FIG. 5). The bands for both forms of the protein were detected in samples from the third cycle, starting at the 6h time point and later. Bands were not detected on day 1 of the fourth cycle (not shown). One patient showed no LC3B bands, although control CD9 bands for the same membrane revealed uniform bands at all time points (FIG. 6). Variations in results are attributed to different patient diagnoses and prior treatments. However, it is noted that LC3B-I and LC3B-II exhibit some instability, such that lack of detection might also be attributed to handling errors.

A third group of patients (n=3) received a dose of 150 mg/day CQ, in addition to the Gem/Carb regimen. CQ administration began on day −7 of the treatment cycle and continued throughout. This group had a lower tolerance to the treatment, compared to other groups. Only one candidate completed the 4 cycles of chemotherapy, with two patients stopping treatment in the first cycle. Looking at patient history, it is believed that the intolerance to treatment is attributable to long history of prior cancer treatments. The patient who completed all 4 cycles of treatment did not exhibit bands for LC3B-II. However, one patient who did not complete treatment showed clear LC3B-II bands via western blot (FIG. 7). This patient showed LC3B-II bands beginning at 24h. Results show LC3B-II is detectable in exosomes extracted from patient plasma samples, without the need for invasive biopsies.

### Example 5. Cell Survival with Combination of Gem/Carb+CQ

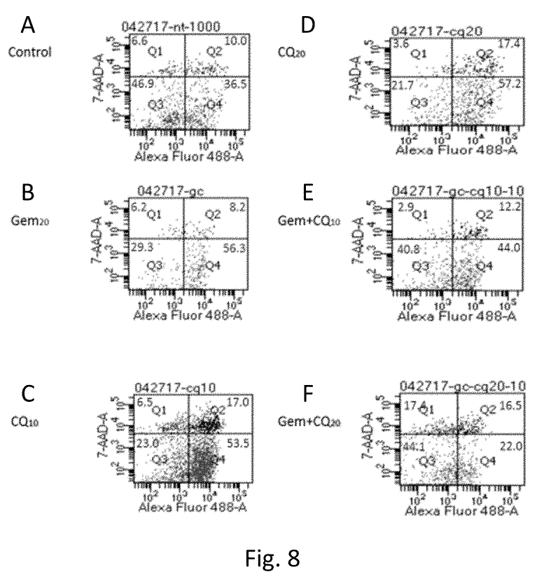

Previous reports suggest Gem/Carb regimen has a stimulatory effect on autophagy. The effect of autophagy inhibitor CQ was examined via a cell survival assay by flow cytometry using annexin V (FIG. 8).

HEK293 cells were plated in 6 well cell culture plates and treated with control (A), Gem 20 μM (B), CQ 10 μM (C), CQ 20 μM (D), Gem 20 μM+CQ 10 μM (E), and Gem 20 μM+CQ 20 μM (F). Cells treated with CQ at both concentrations had increased expression of phosphatidyl serine compared to control from 10% up to almost 17% (C,E). Cells treated with Gem 20 μM alone had only 8.2% apoptotic cells (B). The main cell population in Gem 20 μM-treated cells was in early apoptotic phase.

Comparing single agent treatments with combination treatments, changes in the distribution of the cell population are evident. Combination treatment yielded an increase in the apoptotic cell percentage from 10% to 12.2% and 16.5% compared to control with Gem+CQ10 (E) and Gem+CQ20 (F) concentrations, respectively. These percentages were still higher than Gem20-only treated cells (8.2%, (B)). Further, the effect was dose-dependent, as increasing CQ treatment concentration increased PS-expressing percentage of cells. It might be argued that the combination treatment percentages of apoptotic cells (E,F) were less than that for CQ-only treated cells (D), as the combination treated cells maintained approximately 17% apoptotic cells for both concentrations (E, F). The fraction of necrotic cells was comparably high in Gem+CQ20 cells (F). This suggests that increasing the dose of CQ with Gem implicated necrosis as a process to affect tumor cells. Study results indicate that the addition of CQ likely increased the anti-cancer benefit from the chemotherapeutic agents Gem and Carb.

All documents cited are incorporated herein by reference; the citation of any document is not to be construed as an admission that it is prior art with respect to the present invention.

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A method of detecting microtubule-associated protein 1 light chain 3 B (LC3B) expression as a marker for autophagy in a subject, the method comprising:
obtaining a liquid biological sample from the subject;
isolating exosomes from the liquid biological sample; and
detecting LC3B expression in the isolated exosomes.

2. The method of claim 1, wherein the liquid biological sample is selected from the group consisting of peripheral blood, ascites, and urine.

3. The method of claim 2, wherein the liquid biological sample is peripheral blood.

4. The method of claim 1, wherein isolating exosomes from the liquid biological sample comprises:
centrifuging the liquid biological sample to provide a supernatant and a pellet comprising cells and cellular debris;
treating the supernatant with an exosome extraction reagent to precipitate the exosomes;
centrifuging the treated supernatant to provide a pellet comprising the exosomes; and
resuspending the exosomes in phosphate buffered saline.

5. The method according to claim 1, wherein detecting LC3B expression in the isolated exosomes comprises carrying out a western blot.

6. The method according to claim 5, wherein the western blot is performed using an anti-LC3B monoclonal antibody or polyclonal antibody.

7. The method according to claim 6, wherein the western blot is performed using a goat, rabbit, or mouse antibody.

8. The method according to claim 1, further comprising the step of quantifying LC3B expression in the isolated exosomes.

9. The method according to claim 1, wherein detecting LC3B expression comprises detecting expression of one or more of LC3B-I and LC3B-II.

10. The method of claim 1, wherein the subject is a human.

11. The method of claim 1, wherein the subject is suffering from a solid malignancy.

12. The method of claim 11, wherein the method does not include obtaining a biopsy of the solid malignancy.

13. A method of detecting microtubule-associated protein 1 light chain 3 B (LC3B) expression as a marker for autophagy in a subject suffering from a solid malignancy, the method comprising:
obtaining a peripheral blood sample from the subject;
isolating exosomes from the peripheral blood sample; and
detecting LC3B expression in the isolated exosomes,
wherein the method does not include obtaining a biopsy of the solid malignancy.

14. The method of claim 13, wherein isolating exosomes from the peripheral blood sample comprises:
centrifuging the peripheral blood sample to provide a supernatant and a pellet comprising cells and cellular debris;
treating the supernatant with an exosome extraction reagent to precipitate the exosomes;
centrifuging the treated supernatant to provide a pellet comprising the exosomes; and
resuspending the exosomes in phosphate buffered saline.

15. The method according to claim 13, wherein detecting LC3B expression in the isolated exosomes comprises carrying out a western blot assay.

16. The method according to claim 15, wherein the western blot is performed using an anti-LC3B monoclonal antibody or polyclonal antibody.

17. The method according to claim 15, wherein the western blot is performed using a goat, rabbit, or mouse antibody.

18. The method according to claim 13, wherein detecting LC3B expression comprises detecting expression of one or more of LC3B-I and LC3B-II.

19. The method of claim 13, wherein the subject is a human.

20. The method according to claim 13, further comprising the step of quantifying LC3B expression in the isolated exosomes.
